(19) United States
(12) Patent Application Publication
(10) Pub. No.: US 2025/0135144 A1
(43) Pub. Date: May 1, 2025

(54) A POSITIONING AND STABILISING STRUCTURE FOR A PATIENT INTERFACE

(71) Applicant: ResMed Asia Pte. Ltd., Singapore (SG)

(72) Inventors: Thontira SUPAOPASPHUN, Singapore (SG); Mohankumar Krishnan VALIYAMBATH, Singapore (SG); Bangzheng TAN, Singapore (SG)

(21) Appl. No.: 18/692,784

(22) PCT Filed: Sep. 16, 2022
(86) PCT No.: PCT/SG2022/050668

§ 371 Date: Mar. 15, 2024

(30) Foreign Application Priority Data
Sep. 17, 2021 (SG) 10202110296S

(51) Int. Cl. A61M 16/06; A61M 16/08

(52) U.S. Cl. CPC A61M 16/0683; A61M 16/0611; A61M 16/0616; A61M 16/0883

(57) ABSTRACT

A positioning and stabilising structure for a patient interface comprises a headgear portion, the headgear portion comprising a first headgear portion and a second headgear portion, wherein the first headgear portion and the second headgear portion are joined by a stitchless joint comprising at least one polymer layer spanning between the first headgear portion and the second headgear portion and applied to the first headgear portion and the second headgear portion.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534.

Another technique is the use of one or more straps and/or stabilising harnesses. Many such harnesses suffer from being one or more of ill-fitting, bulky, uncomfortable and awkward to use.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2: 2007, 10 cmH2O
pressure at 1 m)

| | | A-weighted | A-weighted | |
|---|---|---|---|---|
| | | sound power | sound pressure | |
| | | level dB(A) | dB(A) | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal pillows | 37 | 29 | 2004 |
| Mirage | | | | |
| SwiftTM (*) | | | | |
| ResMed | nasal pillows | 28 (3) | 20 (3) | 2005 |
| Mirage | | | | |
| SwiftTM II | | | | |
| ResMed | nasal pillows | 25 (3) | 17 (3) | 2008 |
| Mirage | | | | |
| SwiftTM LT | | | | |
| ResMed AirFit | nasal pillows | 21 (3) | 13 (3) | 2014 |
| P10 | | | | |

(*one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m distance |
| Walter Broadly Litter Hog: | | |
| B+ Grade | | |
| Conversational speech | 60 | 1 m distance |
| Average home | 50 | |
| Quiet library | 40 | |
| Quiet bedroom at night | 30 | |
| Background in TV studio | 20 | |

### 1.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure to provide a force to hold a seal-forming structure of a patient interface in a therapeutically effective position on a patient's head, the seal-forming structure constructed and arranged to form a seal with a region of the patient's face surrounding an entrance to the patient's airways for sealed delivery of a flow of air at a therapeutic pressure of at least 6 cmH2O above ambient air pressure to at least the patient's nares throughout the patient's respiratory cycle in use, the positioning and stabilising structure comprising:

a headgear portion, the headgear portion comprising a first headgear portion and a second headgear portion, wherein the first headgear portion and the second headgear portion are joined by a stitchless joint comprising at least one polymer layer spanning between the first headgear portion and the second headgear portion and applied to the first headgear portion and the second headgear portion.

In examples: (a) adjacent edges of the first headgear portion and the second headgear portion joined by the stitchless joint may be non-overlapping; (b) the adjacent edges of the first headgear portion and the second headgear portion joined by the stitchless joint may be abutting when the positioning and stabilising structure is unloaded; (c) the adjacent edges of the first headgear portion and the second headgear portion may be directly joined together in addition to the at least one polymer layer; (d) the adjacent edges of the first headgear portion and the second headgear portion may be welded together; (e) the adjacent edges of the first headgear portion and the second headgear portion are not directly joined.

In further examples: (a) the first headgear portion and the second headgear portion may be strap portions; (b) the first headgear portion and the second headgear portion may be headgear conduit portions; (c) one of the first headgear portion and the second headgear portion may be a strap portion, and the other one of the first headgear portion and the second headgear portion may be a headgear conduit portion; (d) one of the first headgear portion and the second headgear portion may be a strap connection tab portion, and the other one of the first headgear portion and the second headgear portion may be a headgear conduit portion.

In further examples: (a) the at least one polymer layer surrounds the first headgear portion and the second headgear portion at the stitchless joint; (b) the at least one polymer layer comprises a first polymer layer applied to a first side of the first headgear portion and the second headgear portion, and a second polymer layer applied to a second side of the first headgear portion and the second headgear portion.

In further examples: (a) the at least one polymer layer comprises an adhesive film material; (b) the at least one polymer layer comprises a thermoplastic material; (c) the at least one polymer layer comprises a thermoplastic elastomer material; (d) the at least one polymer layer comprises a material made of polyamide, polyester, polyethylene, polyurethane, polyolefin, vinyl, nylon, ethylene or any suitable combination thereof; (e) the at least one polymer layer comprises a polyurethane material; (e) the at least one polymer layer comprises a thermoplastic polyurethane material.

In further examples: (a) the at least one polymer layer is exposed to treatment conditions to secure the at least one polymer layer to the first headgear portion and the second headgear portion; (b) the treatment conditions include one or more of: increased heat or temperature conditions, increased pressure conditions, and/or radiation exposure conditions.

In further examples: (a) the at least one polymer layer overlaps each of the first headgear portion and the second headgear portion by a minimum distance to resist a tension force of at least 20 N across the joint; (b) the minimum distance is about 3 mm; (c) the at least one polymer layer overlaps each of the first headgear portion and the second headgear portion by a minimum distance to resist a tension force of at least 40 N; (d) the minimum distance is about 5 mm.

In further examples: (a) the at least one polymer layer may be used to impart different properties to the headgear; (b) the at least one polymer layer may increase stretchability of the headgear across the joint; (c) the at least one polymer layer may impart shape holding properties of the headgear; (d) the at least one polymer layer may impart increased rigidity to at least a portion of the headgear.

In further examples at least one of the first headgear portion and the second headgear portion are constructed at least partially from a textile material.

In examples the positioning and stabilising structure comprises: (a) at least one gas delivery tube to receive the flow of air from a connection port on top of the patient's head and to deliver the flow of air to the entrance of the patient's airways via the seal-forming structure, the gas delivery tube being constructed and arranged to contact, in use, at least a region of the patient's head superior to an otobasion superior of the patient's head, (b) the at least one gas delivery tube comprises a pair of headgear conduits to receive the flow of air from a connection port on top of the patient's head and to deliver the flow of air to the entrance of the patient's airways via the seal-forming structure, each headgear conduit constructed and arranged to contact, in use, at least a region of the patient's head superior to an otobasion superior of the patient's head on a respective side of the patient's head; and a headgear strap. In examples a flexible cover may be provided to at least a portion of each headgear conduit. In examples the flexible cover comprises a textile. In examples: (a) a stitchless joint comprising at least one polymer layer may be provided between a portion of the headgear conduit and a portion of the headgear strap; (b) a stitchless joint comprising at least one polymer layer may be provided between a first portion of the headgear conduit and a second portion of the headgear conduit.

In further examples the headgear strap comprises: 1) a ring strap portion having a superior portion configured to overlay the parietal bones of the patient's head in use and having an inferior portion configured to overlay or lie inferior to the occipital bone of the patient's head in use, the ring strap portion defining a loop, 2) a pair of superior strap portions, each configured to connect between the ring strap portion and a mask portion of the patient interface in use on a respective side of the patient's head superior to an otobasion superior, 3) a pair of inferior strap portions, each inferior strap portion configured to connect between the ring strap portion and the mask portion of the patient interface on a respective side of the patient's head inferior to the otobasion superior. In examples, a stitchless joint comprising at least one polymer layer may be provided between two or more of the strap portions.

In further examples the headgear strap comprises: 1) a back strap portion configured to overlay the occipital bone of the patient's or lie inferior to the occipital bone of the patient's head in use; 2) a pair of superior strap portions, each configured to connect between the back strap portion and a respective headgear conduit on a respective side of the patient's head; and 3) a pair of inferior strap portions, each configured to connect between the back strap portion and a mask portion of the patient interface. In examples, a stitchless joint comprising at least one polymer layer may be provided between two or more of the strap portions.

Another aspect of one form of the present technology is a patent interface, comprising: a seal-forming structure constructed and arranged to form a seal with a region of a patient's face surrounding an entrance to the patient's airways for sealed delivery of a flow of air at a therapeutic pressure to at least the patient's nares throughout the patient's respiratory cycle in use; and a positioning and stabilising structure to provide a force to hold the seal-forming structure in a therapeutically effective position on the patient's head, wherein the positioning and stabilising structure is substantially as described herein.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

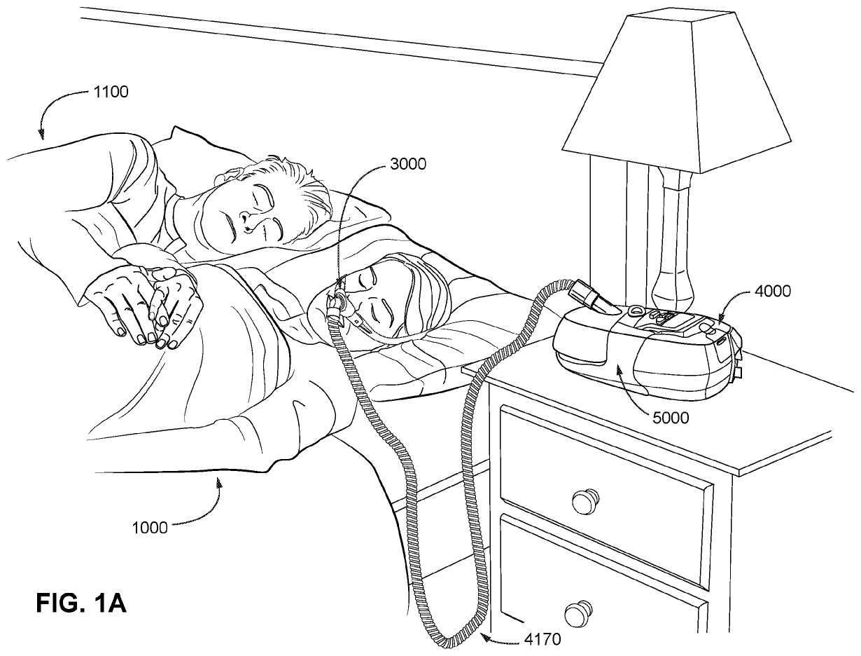

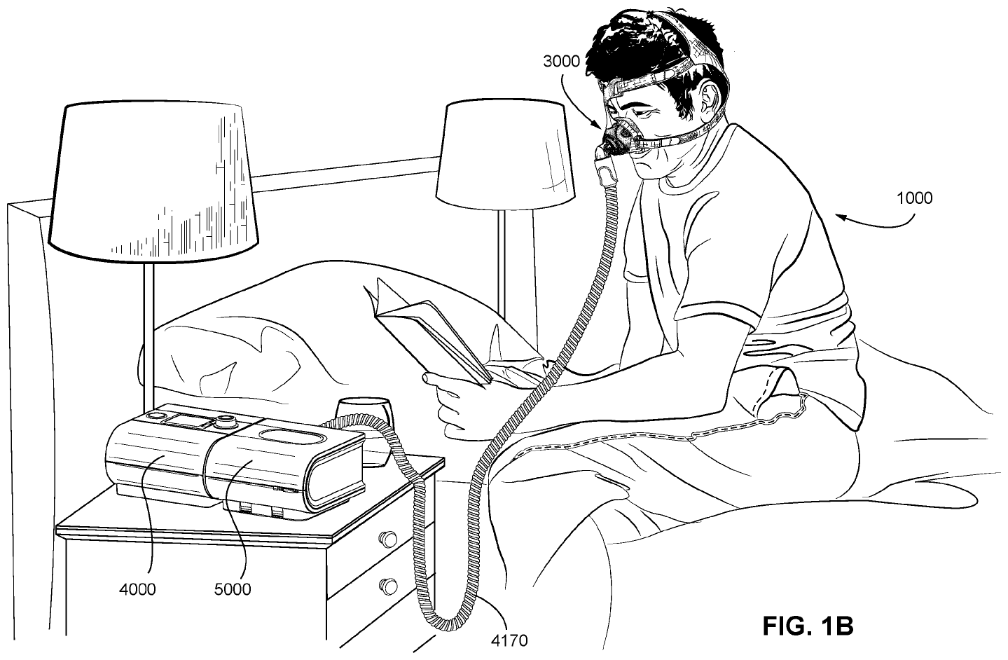

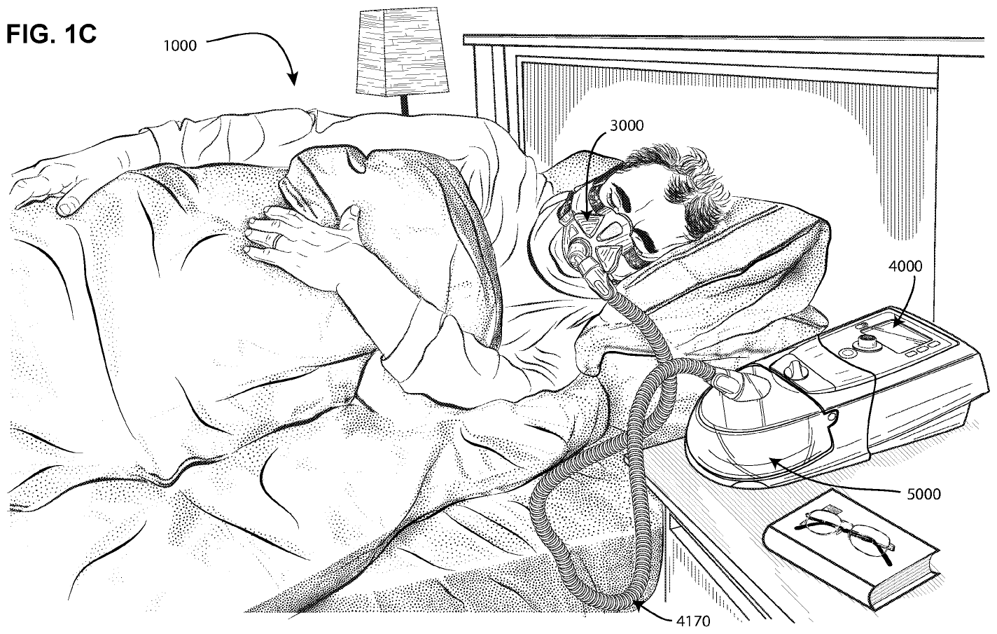

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

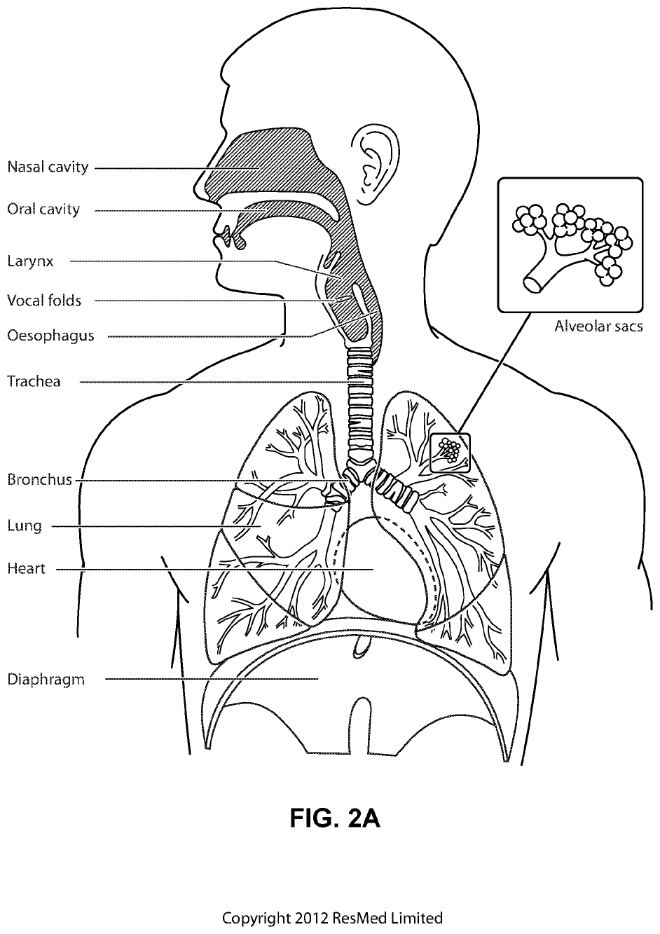

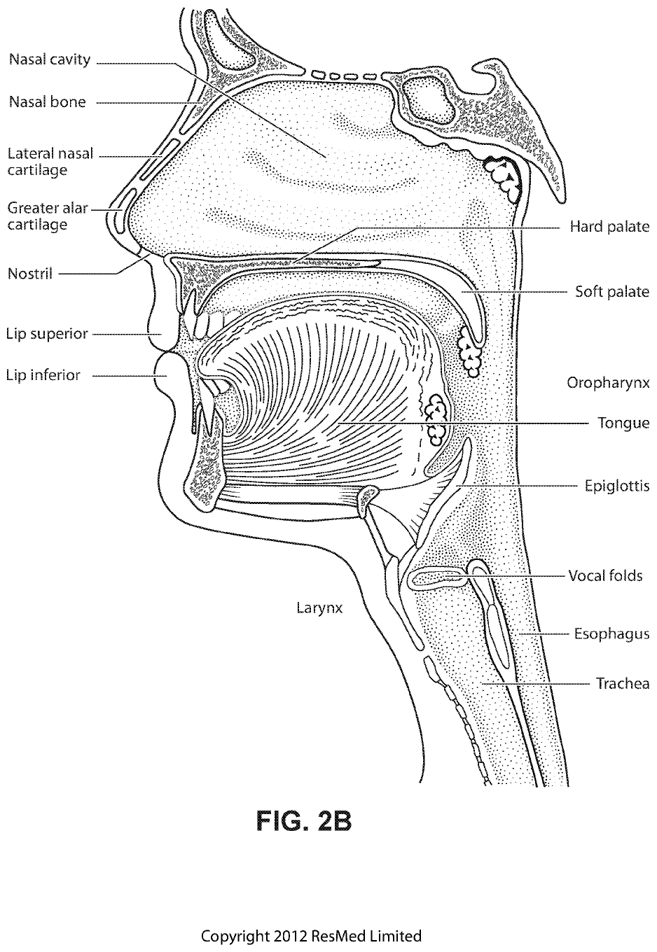

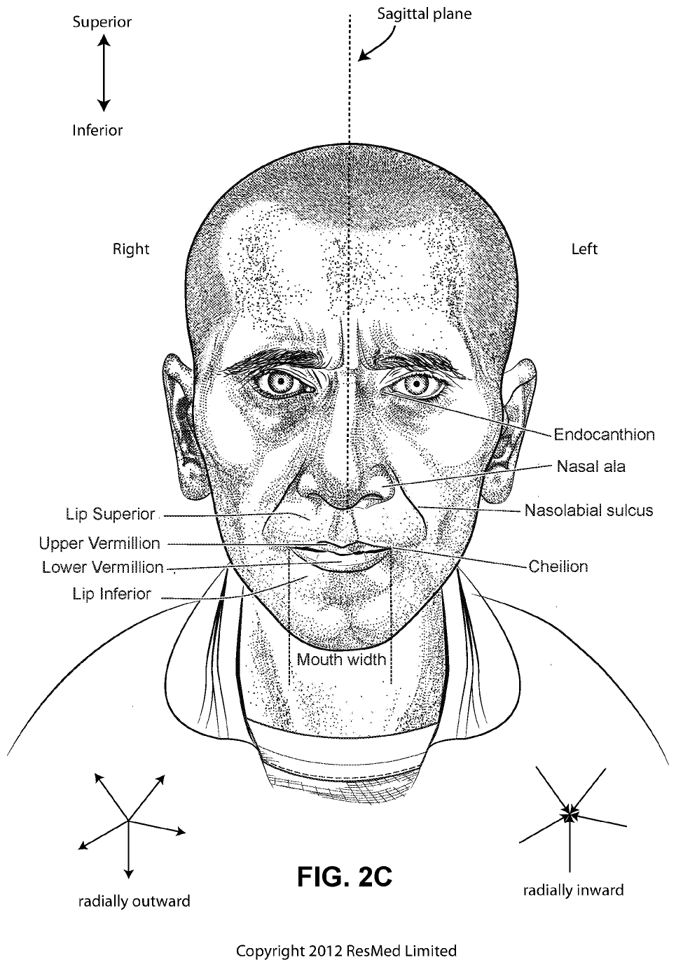

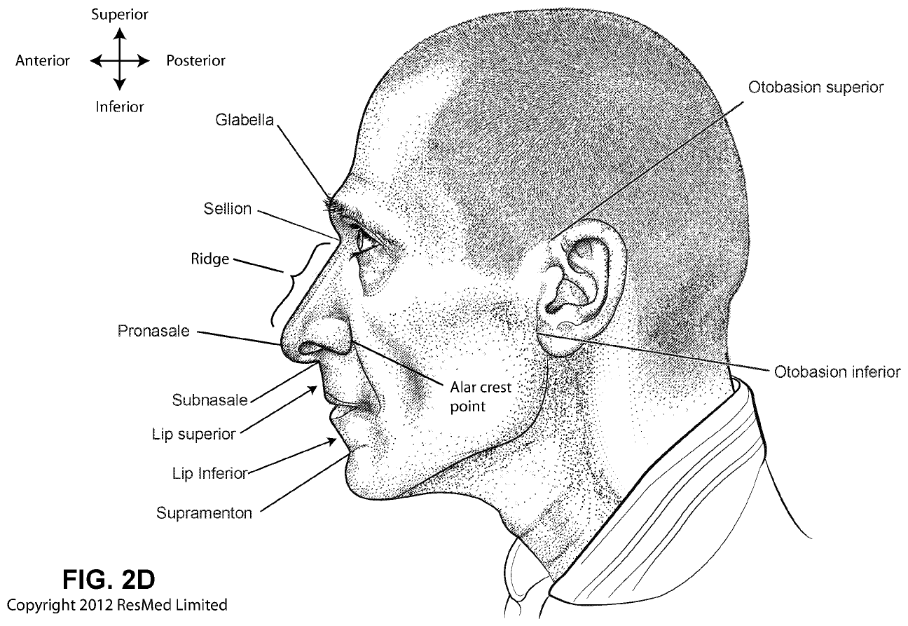

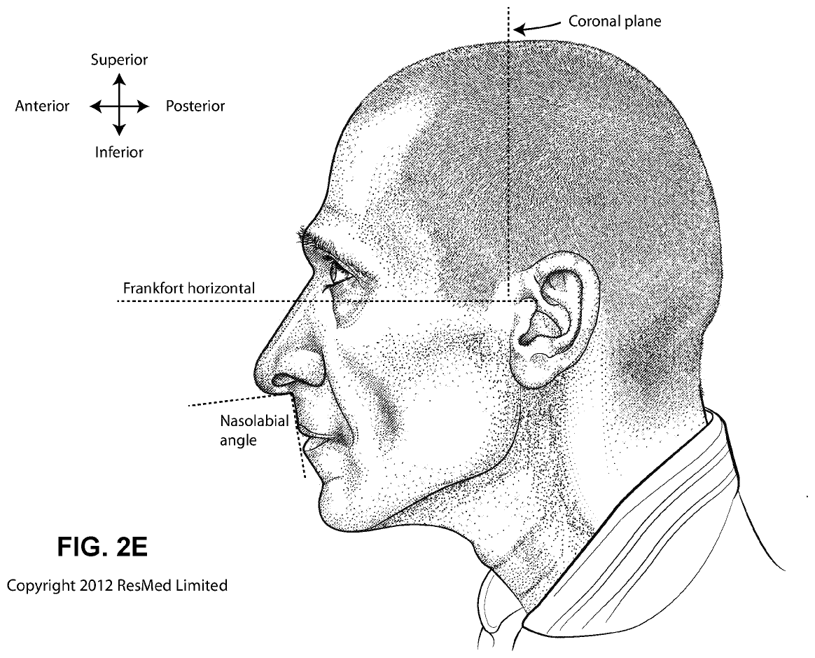

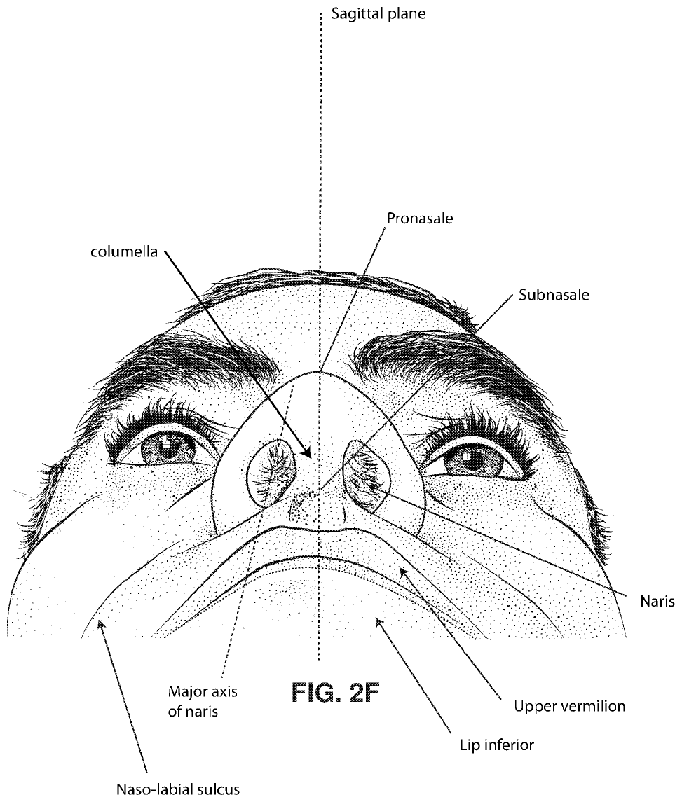

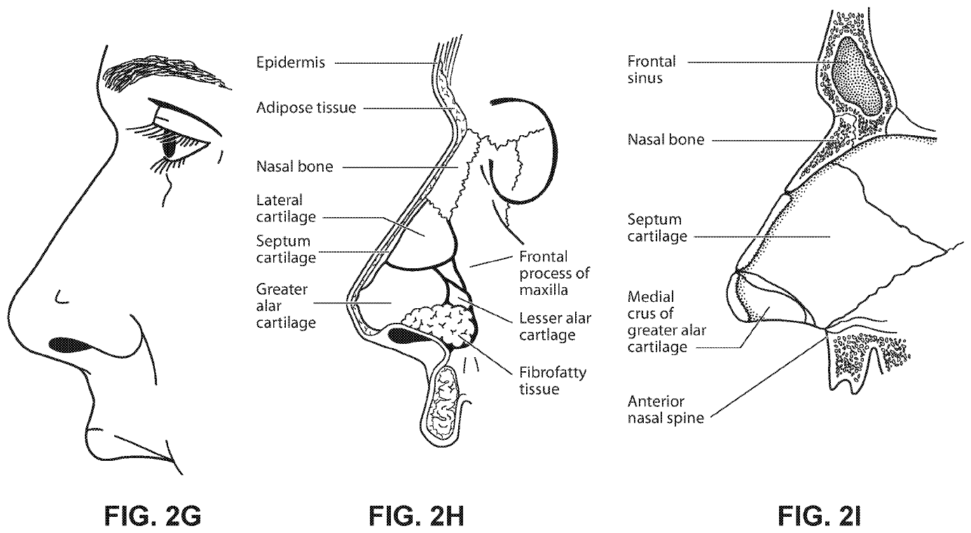

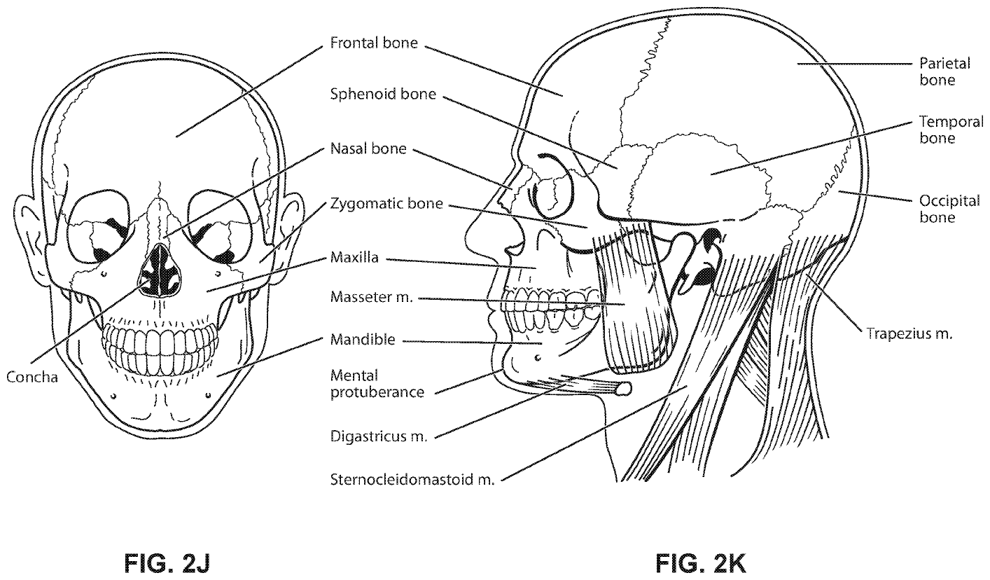

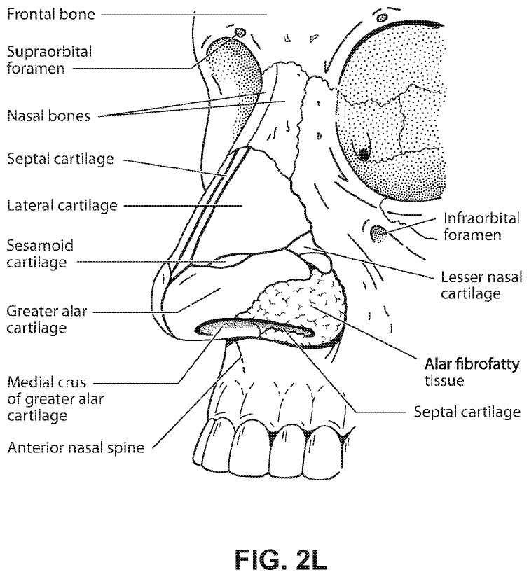

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2L shows an anterolateral view of a nose.

### 3.3 Patient Interface

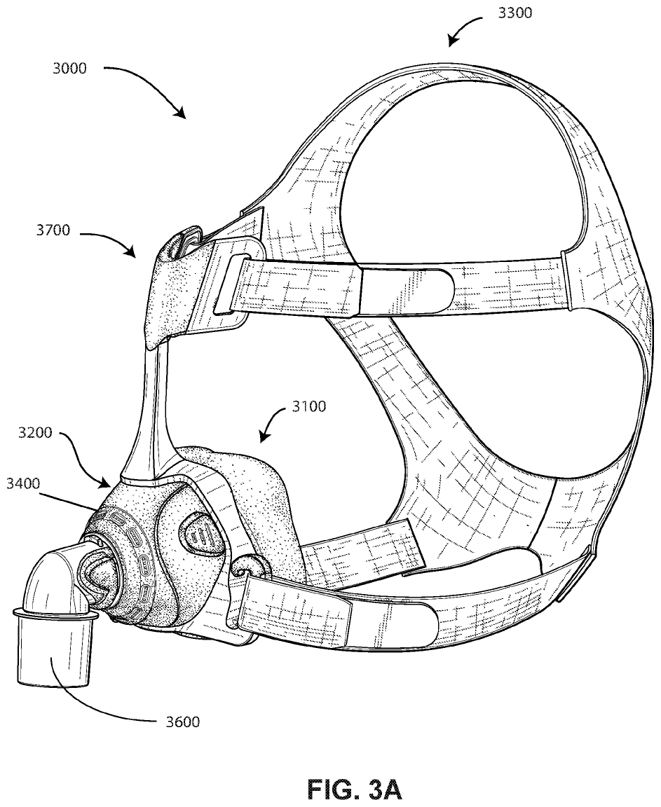

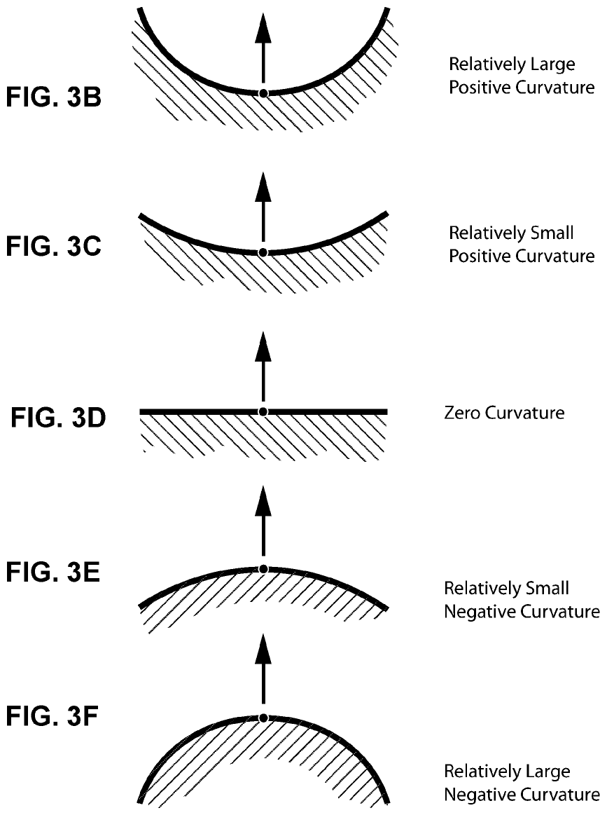

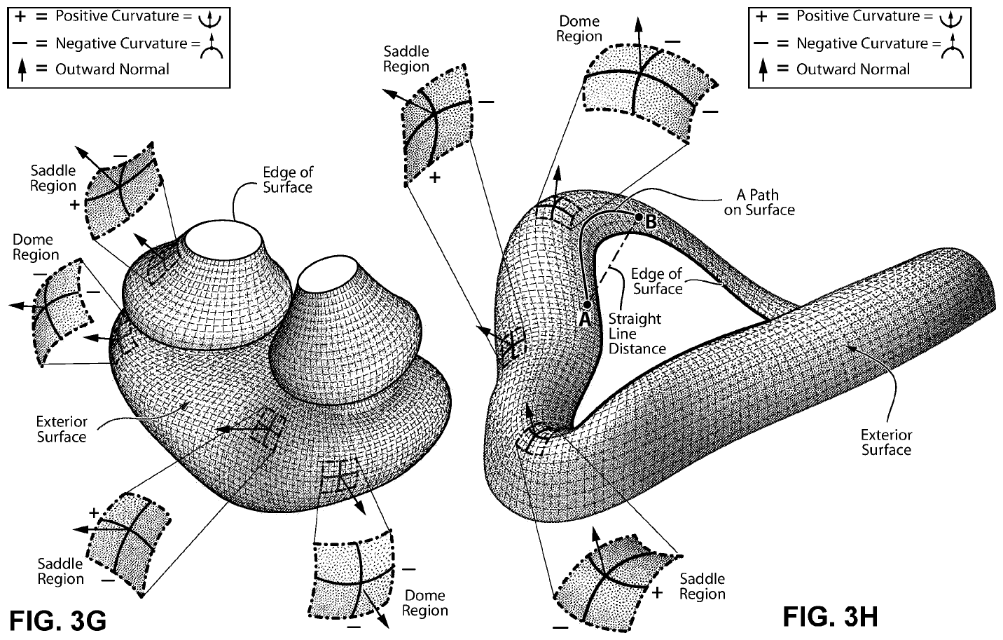

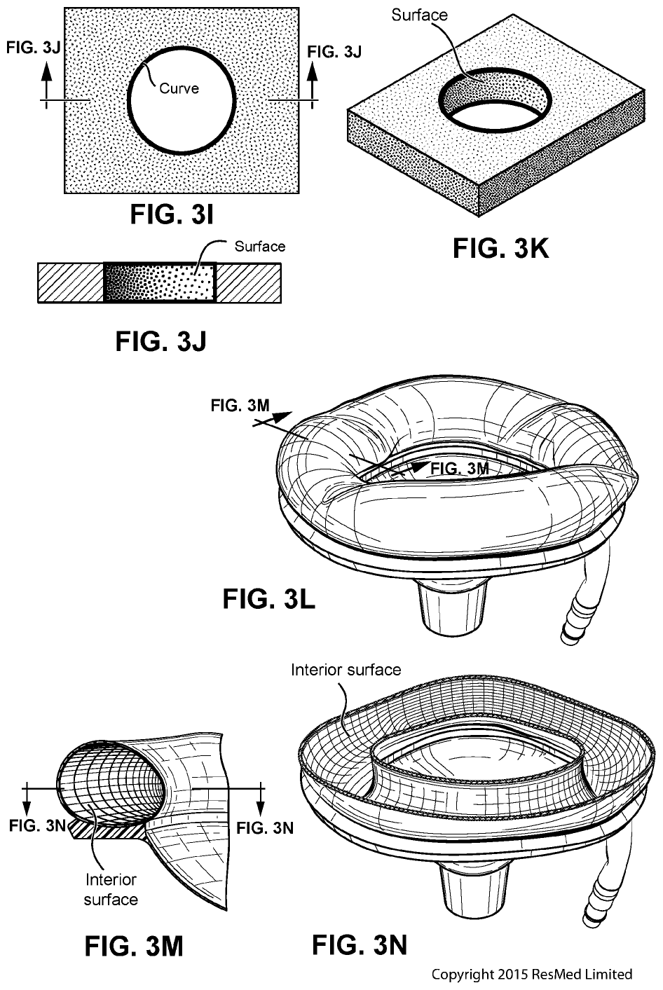

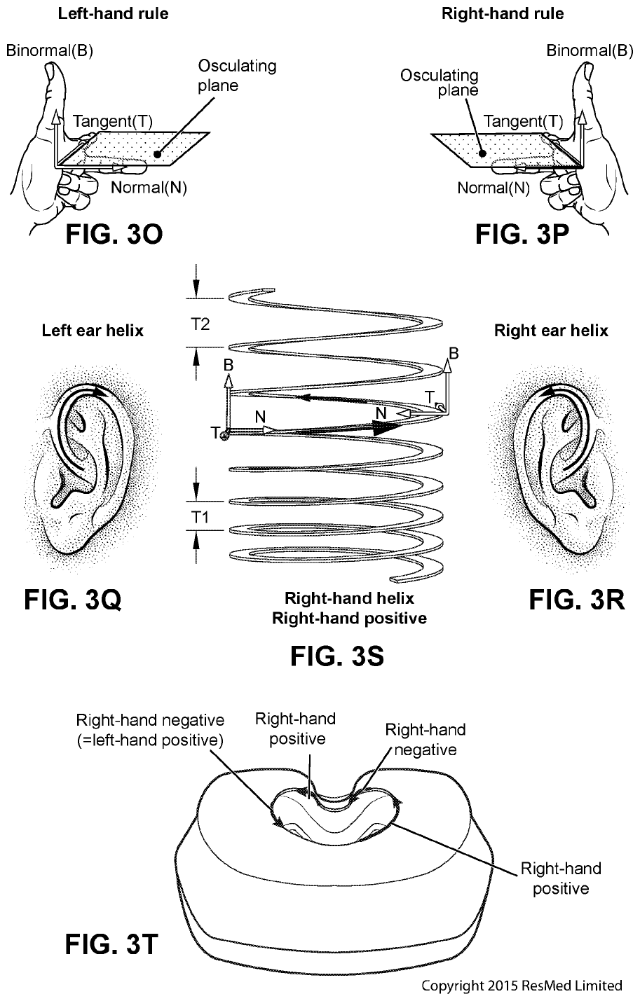

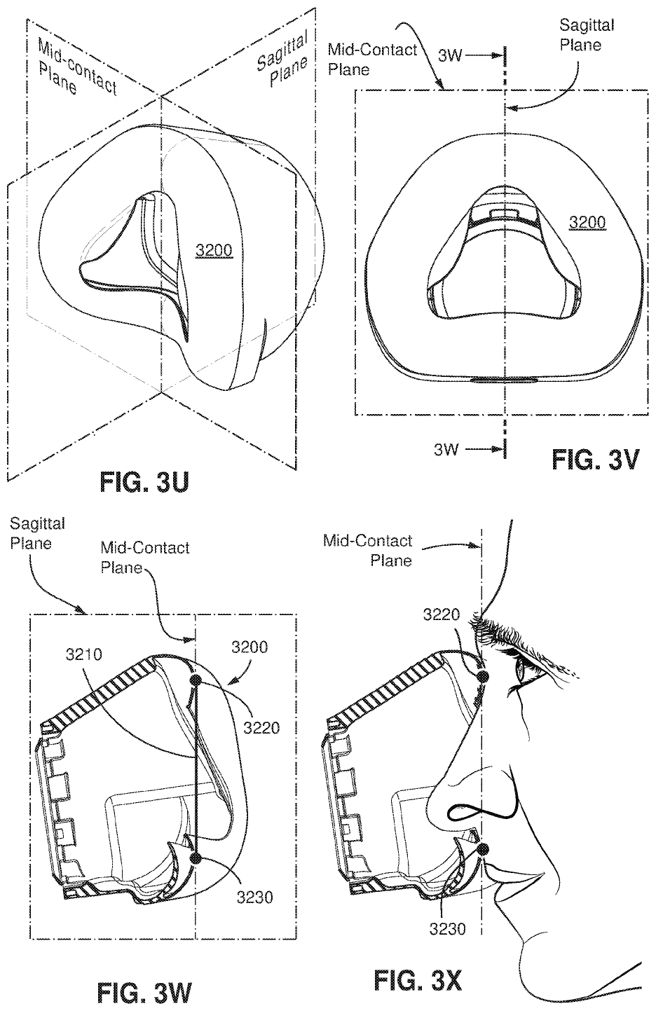

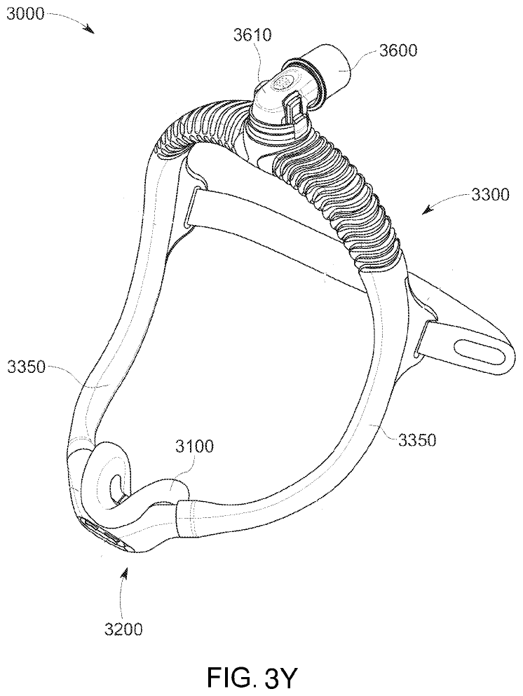

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a mask having conduit headgear in accordance with one form of the present technology.

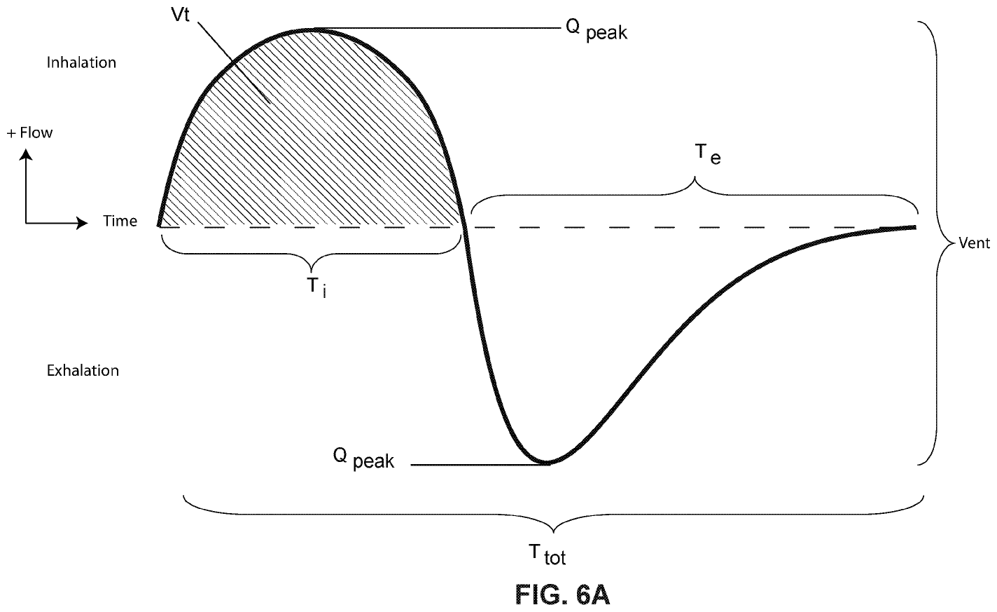

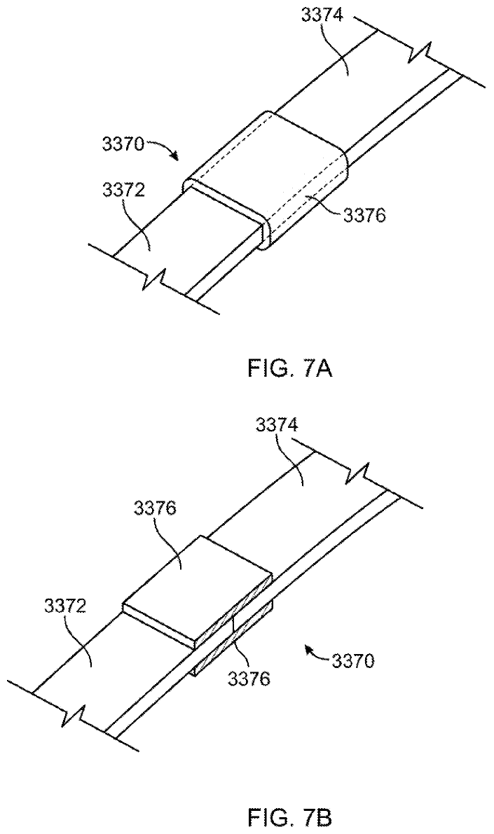

FIG. 7A shows a first form of a stitchless joint between two portions of a headgear in accordance with one form of the present technology.

FIG. 7B shows a second form of a stitchless joint between two portions of a headgear in accordance with one form of the present technology.

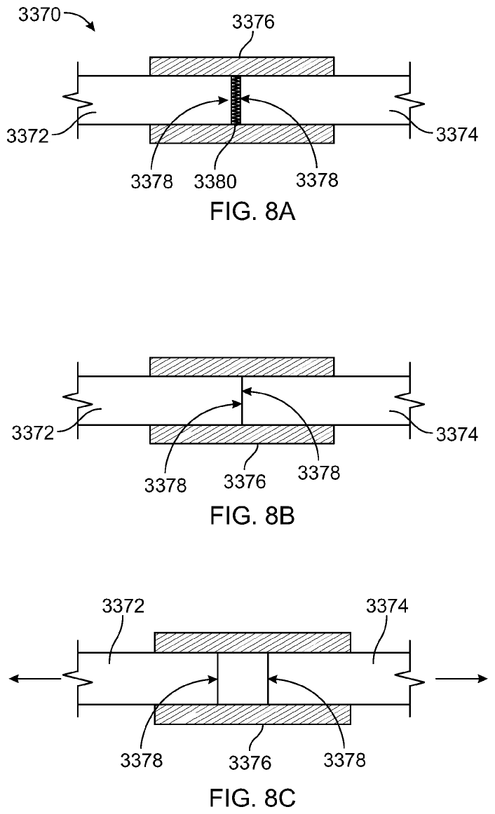

FIG. 8A shows a cross section of a stitchless joint between two welded portions of a headgear in accordance with one form of the present technology.

FIG. 8B shows a cross section of a stitchless joint between two portions of a headgear in a released condition, in accordance with one form of the present technology.

FIG. 8C shows a cross section of the stitchless joint in which the two portions of the headgear are drawn apart under tension.

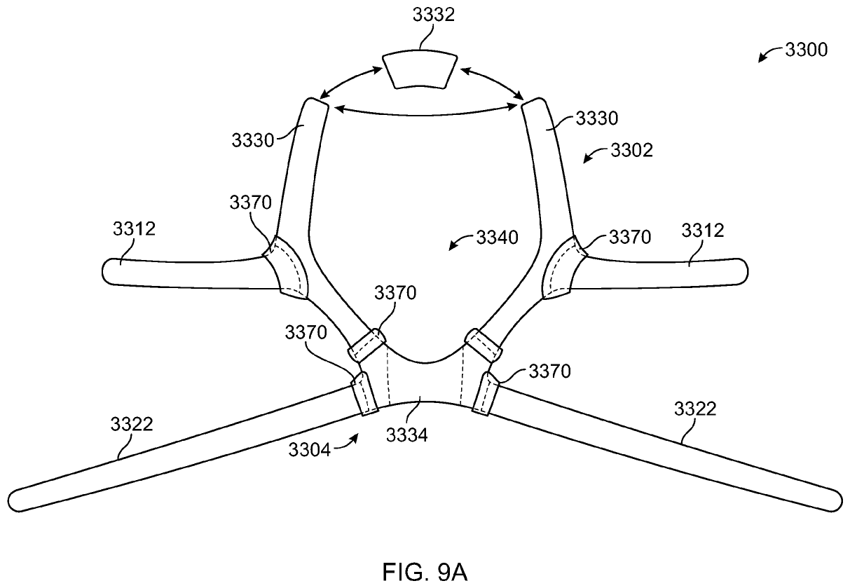

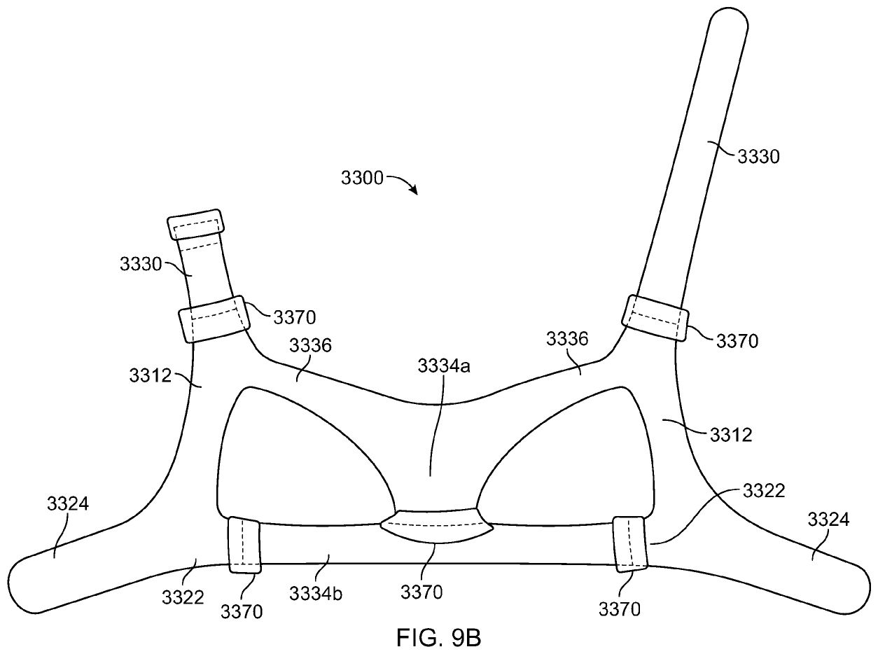

FIG. 9A shows a positioning and stabilising portion 3300 according to a form of the present technology.

FIG. 9B shows a positioning and stabilising portion 3300 according to another form of the present technology.

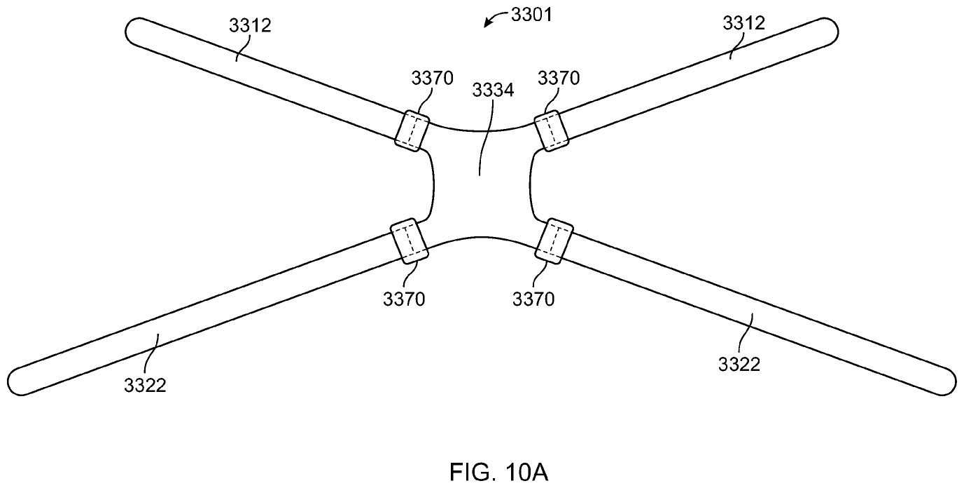

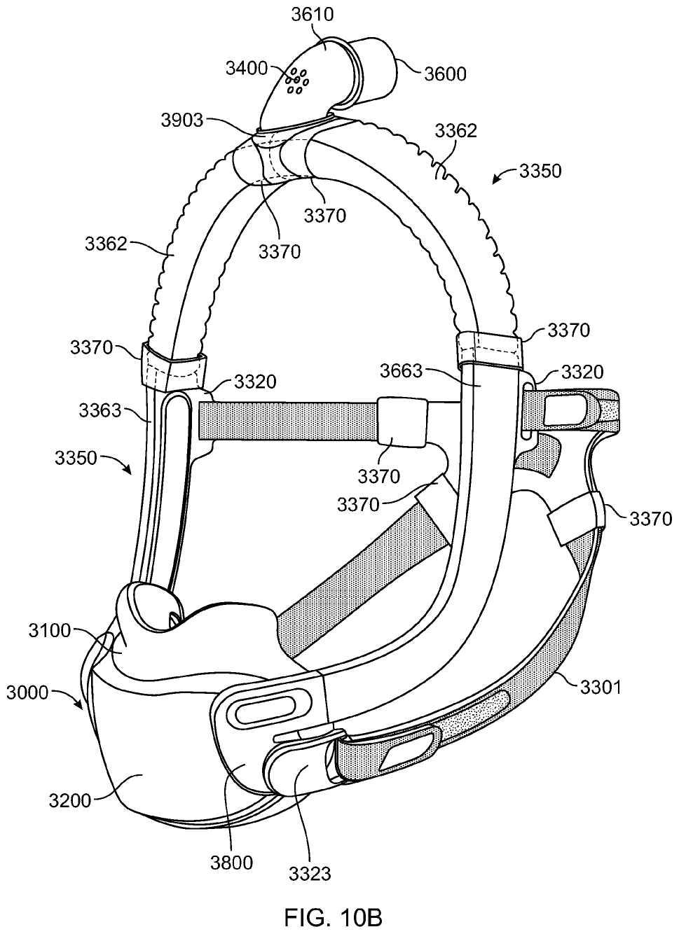

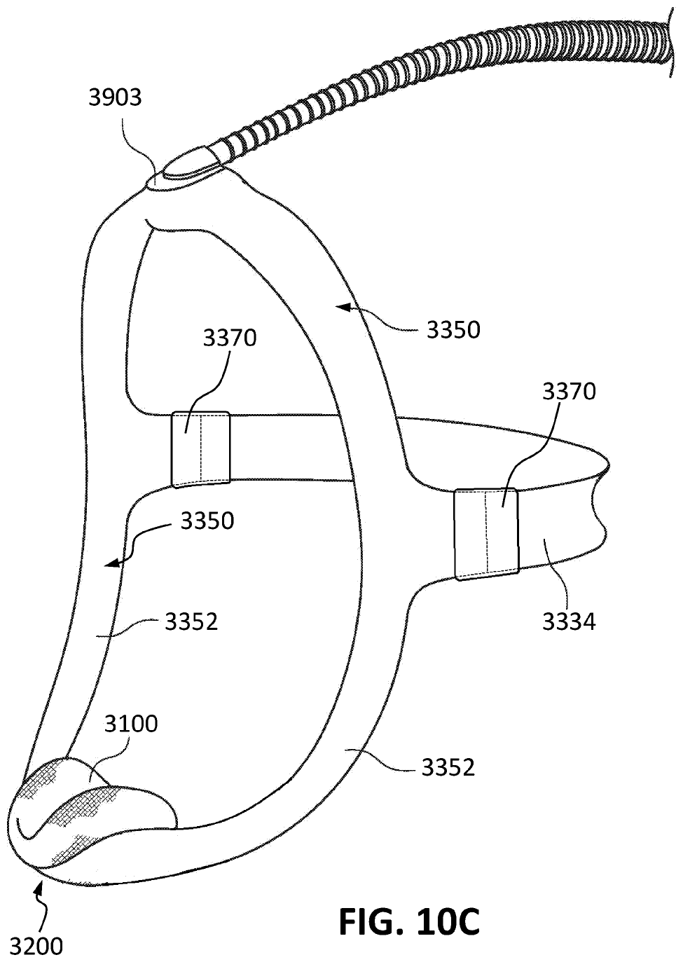

FIG. 10A shows a positioning and stabilising portion 3300 according to another form of the present technology.

FIG. 10B shows a patient interface comprising a positioning and stabilising portion 3300 according to another form of the present technology.

FIG. 10C shows a patient interface comprising a positioning and stabilising portion 3300 according to another form of the present technology.

### 3.4 RPT Device

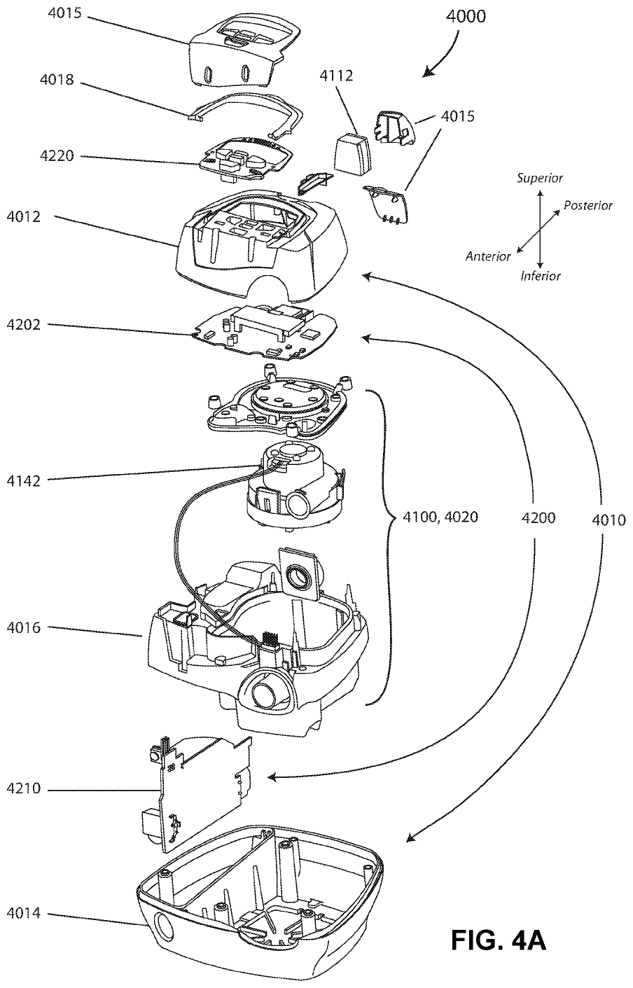

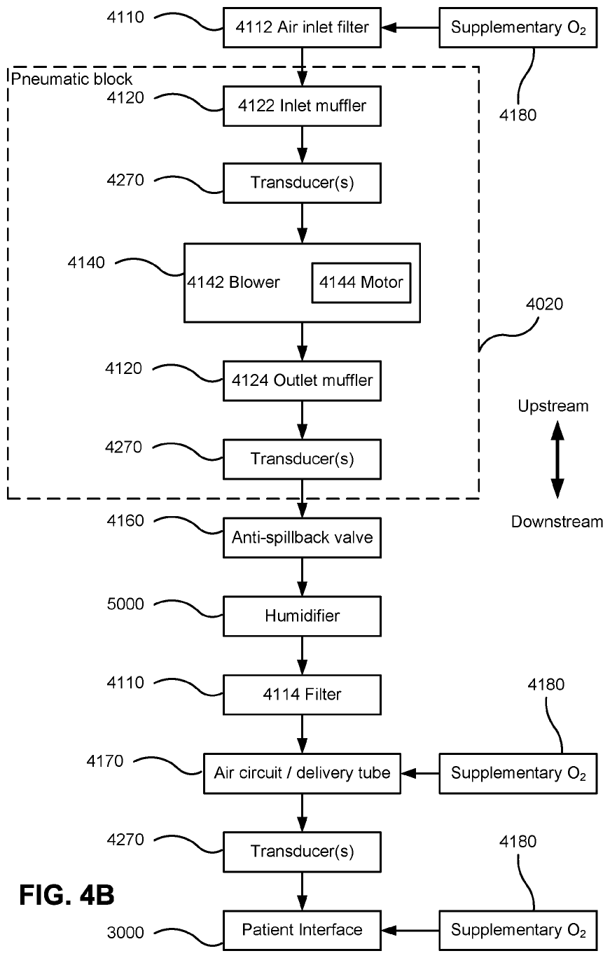

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

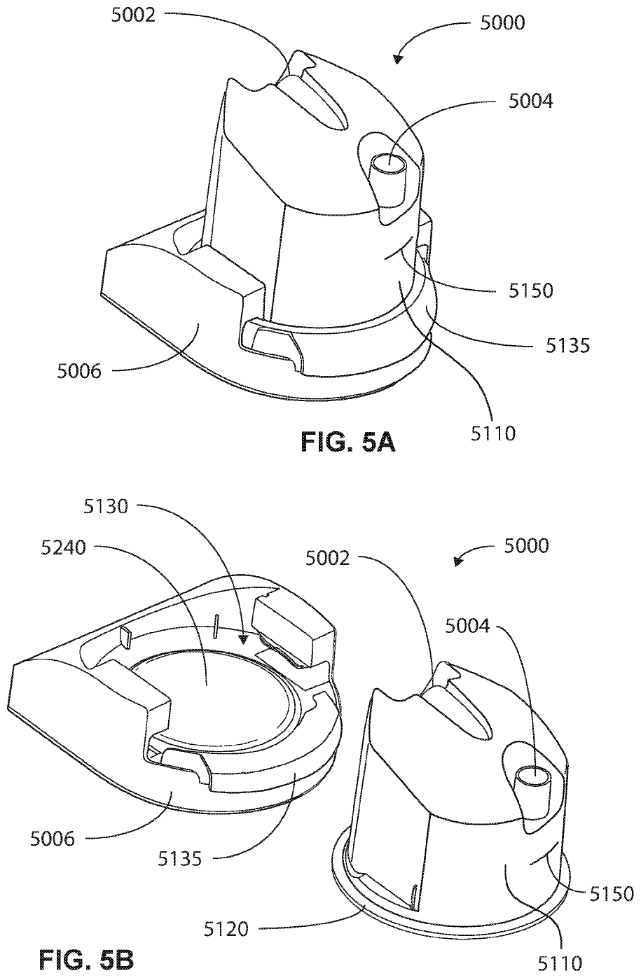

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Breathing Waveforms

FIG. 6A shows a model typical breath waveform of a person while sleeping.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Y, a non-invasive patient interface 3000 in accordance with another aspect of the present technology comprises the following functional aspects: a seal-forming structure 3000, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components in the sense that it or they can be replaced with different components, for example components of a different size.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 4.3.3.1 Conduit Headgear

### 4.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3Y, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes. In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head.

In the form of the technology shown in FIG. 3Y the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used they may be indirectly connected together, for example each may be connected to a T-shaped connector having two arms/branches each fluidly connectable to a respective one of the tubes 3350 and a third arm or opening in the T-shaped connector providing the connection port 3600 for fluid connection to the air circuit 4170 in use.

### 4.3.3.2 Particular Examples of the Present Technology

One form of the present technology comprises a positioning and stabilising structure 3300 to provide a force to hold a seal-forming structure of a patient interface in a therapeutically effective position on a patient's head, the seal-forming structure constructed and arranged to form a seal with a region of the patient's face surrounding an entrance to the patient's airways for sealed delivery of a flow of air at a therapeutic pressure of at least 6 cmH2O above ambient air pressure to at least the patient's nares throughout the patient's respiratory cycle in use, the positioning and stabilising structure comprising a headgear portion, the headgear portion comprising a first headgear portion and a second headgear portion, wherein the first headgear portion and the second headgear portion are joined by a stitchless joint comprising at least one polymer layer spanning between the first headgear portion and the second headgear portion and applied to the first headgear portion and the second headgear portion.

In one form of the present technology, the polymer layer comprises material suitable for engaging with the respective headgear portions. In examples the polymer layer may comprise a thermoplastic material. For example, the at least one polymer layer comprises a thermoplastic elastomer material. For example, the at least one polymer layer comprises a thermoplastic polyurethane material. However, it should be appreciated that other suitable materials are contemplated. For example, the at least one polymer layer may comprise a material made of polyamide, polyester, polyethylene, polyurethane, polyolefin, vinyl, nylon, ethylene or any suitable combination thereof. Examples of a suitable polymer layer include polymer heat seal seam tapes manufactured by Bemis Associates, Inc. (USA). These seam tapes are thermoplastic polymers that may be applied by commercially available taping machines and join elements formed of a variety of materials, particularly textile materials.

The polymer layer may be applied to the headgear portions using known manufacturing techniques—for example exposure to treatment conditions including one or more of: increased heat or temperature conditions, increased pressure conditions, and/or radiation exposure conditions. In general, the application of heat and/or pressure induces the polymer layer to soften or melt so as to infiltrate the material of the headgear portions. Upon subsequent cooling, the polymer layer becomes securely bonded to each of headgear portions, thereby holding the headgear portions together. Selection or specification of a material for the at least one polymer layer may depend on the base material to which it is to be provided. In particular, the polymer layer may be selected so as to have a lower softening point than the base material. In examples, the base material may be a fabric, such as a fabric composite of one or more of: nylon, polyethersulfone (PES), blended fabric, cotton, polyester, etc. By way of example, a polymer heat seal seam tape applied using a heat press technique may be subjected to temperatures in the range of 100° C. to 150° C., pressures in the range of 50 psi to 100 psi, for a duration in the range of 10 seconds to 60 seconds. By way of example, a polymer heat seal seam tape applied using a heat welding technique may be subjected to temperatures in the range of 200° C. to 500° C., loads in the range of 50 psi to 100 psi, and air pressure in the range of 3 psi to 8 psi.

In one form of the present technology, as shown in FIG. 7A, a stitchless joint 3370 may be formed between a first headgear portion 3372 and a second headgear portion 3374 where the at least one polymer layer 3376 surrounds the first headgear portion 3372 and the second headgear portion 3374 at the stitchless joint 3370—i.e. encloses the first headgear portion 3372 and the second headgear portion 3374.

In an alternative form of the present technology, as shown in FIG. 7B, a stitchless joint 3370 may be formed between a first headgear portion 3372 and a second headgear portion 3374 where a first polymer layer 3376 is applied to a first side of the first headgear portion 3372 and the second headgear portion 3374, and a second polymer layer 3376 applied to a second side of the first headgear portion 3372 and the second headgear portion 3374.

In one form of the present technology, as shown in FIG. 8A, adjacent edges 3378 of the first headgear portion 3372 and the second headgear portion 3374 joined by the stitchless joint 3370 are directly joined—for example, by an ultrasonic weld 3380.

In an alternative form of the present technology, as shown in FIG. 8B and FIG. 8C, the adjacent edges 3378 are not directly joined—i.e. are joined by the mutual connection to the polymer layer 3376. In examples, the adjacent edges 3378 may be substantially abutting when the positioning and stabilising structure is unloaded—i.e. while the patient interface is not worn and the associated tension forces are not being applied. In examples, the polymer layer 3376 may have elastic properties, permitting the adjacent edges 3378 to be drawn apart under tension, and returned to their original position once released.

In one form of the present technology, the distance by which the polymer layer 3376 overlaps each of the first headgear portion 3372 and the second headgear portion 3372 (i.e. the distance from an edge of the polymer layer 3376 to the closest edge 3378 of the first headgear portion 3372 and the second headgear portion 3372) may be determined by a desired joint strength requirement—for example an ability to resist a minimum tension force. For example, a superior strap may have a lower tension force than an inferior strap.

In an example, the minimum distance for the overlap on each portion to resist a tension force of at least 20 N across the joint may be about 3 mm. In an example, the minimum distance for the overlap on each portion to resist a tension force of at least 40 N across the joint may be about 5 mm.

In one form of the present technology, at least one of the first headgear portion and the second headgear portion are constructed at least partially from a textile material. In comparison with stitched joints commonly used in joining textile or fabric materials, the polymer layer may assist in reducing fabric damage, particularly the delamination of fabric, by avoiding (a) perforation of the fabric and (b) stress concentration which occurs at the stitches when pulled. Furthermore, the stitchless joint of the present technology may increase productivity output and provide savings in labour costs (for example, the speed of sewing stitches along 50 inches of straight seam is about 30-40 seconds, compared with about 20-25 seconds using the stitchless joint of the present technology). The polymer layer may provide a flat, smooth, exterior surface to reduce friction against skin of the user (particularly on the face) and improve comfort in comparison with stitched joints which can present a raised hump and create localised points of increased pressure against the skin. In addition, the stitchless joint of the present technology may be more robust than a stitched equivalent, e.g. potentially resisting deformation even after experiencing forces upwards of 100 N. The stitchless joint of the present technology may also reduce weight of the headgear in comparison with traditionally sewn seams, and consequently provide greater comfort.

In examples of the present technology the first headgear portion 3372 and the second headgear portion 3374 are both strap portions. However the stitchless joint 3370 may be used to join various components of headgear in different configurations, for example headgear conduit portions.

### 4.3.3.2.1 Headgear Strap

In one example of the present technology, shown in FIG. 9A, the positioning and stabilising structure 3300 comprises headgear having a ring strap portion 3340. The ring strap portion 3340 encircles a posterior side of the patient's head, providing a strong anchor for other strap portions that connect to the plenum chamber 3200. The ring strap portion 3340 may also be known as a crown portion, a crown strap, a rear/posterior portion or a halo.

In this example of the present technology, the ring strap portion 3340 of the positioning and stabilising structure 3300 comprises a superior portion 3302 and an inferior portion 3304. The superior portion 3302 lies in use against the patient's head over the parietal bones of the patient's head. The inferior portion 3304 is configured to lie against the patient's head over or inferior to the occipital bone of the patient's head in use. As illustrated, the ring strap portion 3340 defines a loop.

In this example, the ring strap portion 3340 comprises a pair of overhead strap portions 3330. The overhead strap portions 3330 may be configured to connect to each other proximate the sagittal plane of the patient's head. In some examples, the two overhead strap portions 3330 may be joined directly, or via an intermediary joining portion 3332.

In this example, the ring strap portion 3340 comprises a back strap portion 3334 configured to overlay, or lie inferior to, the occipital bone of the patient's head in use.

In this example, the positioning and stabilising structure 3300 comprises a pair of superior strap portions 3312. Each of the superior strap portions 3312 is configured to connect between the ring strap portion 3340 and the plenum chamber 3200. In use, each of the superior strap portions 3312 is located alongside the patient's head, on a respective side, superior to an otobasion superior of the patient's head.

In this example, the positioning and stabilising structure 3300 also comprises a pair of inferior strap portions 3322. Each of the inferior strap portions 3322 is configured to connect between the ring strap portion 3340 and plenum chamber 3200. In use, each of the inferior strap portions 3322 is located alongside the patient's head, on a respective side, inferior to an otobasion superior of the patient's head.

Each of the superior strap portions 3312 and the inferior strap portions 3322 may connect to the plenum chamber 3200 either directly or via a frame 3500 of the positioning and stabilising structure 3300.

In accordance with one aspect of the present technology, the various strap portions may be joined using a stitchless joint 3370.

FIG. 9B shows an example of headgear in the form of a two-point connection headgear 3300. The headgear comprises at least one superior strap portion 3312. The superior strap portion 3312 may be configured to overlie the cheek region of the patient's face, preferably the upper cheek region, and extends between the top of the patient's ear and the patient's eye. The headgear further comprises at least one inferior strap portion 3322. The inferior strap portion 3322 may overlie a region of the patient's head below and behind the patient's ear. The superior strap portion 3312 and the inferior strap portion 3322 are connected to an anterior strap portion 3324. The superior strap portion 3312 and the inferior strap portion 3322 join the anterior strap portion 3324 anterior to the patient's ear, in use. In the embodiment shown, the headgear comprises two superior strap portions 3312, two inferior strap portions 3322, and two anterior strap portions 3324, one of each located on each of the left and right hand sides of the patient's skull. The two anterior strap portions 3324 may each be formed integrally with one end of the superior strap portion 3312 and one end of the inferior strap portion 3322. In the embodiment shown, the two superior strap portions 3312 attach to a crown strap portion 3330 and the two inferior strap portions 3322 attach to a back strap portion 3334 (in this example comprising a superior back strap portion 3334a and an inferior back strap portion 3334b). The crown strap portions 3330 are connected to back strap portion 3334a by two posterior connecting portions 3336 and/or each of the superior strap portions 3312 are joined to the inferior strap portions 3324 by the posterior connecting portions 3336.

Each anterior strap portion 3324 is connected or connectable to a connection portion which engages an interfacing portion of the patient interface 3000, for example an ultra-compact full-face mask, which is configured to seal with the mouth and the nose of the patient.

In accordance with one aspect of the present technology, the various strap portions may be joined using a stitchless joint 3370.

FIG. 10A shows an example of a headgear strap 3301 comprising a pair of superior headgear strap portions 3312, each configured to connect to a respective headgear conduit 3350 of the positioning and stabilising structure 3330 located on a respective lateral side of the patient's head in use (see, FIG. 10B). The headgear strap 3301 also comprises a pair of inferior headgear strap portions 3322, each configured to connect to the plenum chamber 3200. The lower headgear strap portions 3322 may connect directly to the plenum chamber 3200, or via a connector 3800 as illustrated.

The pair of superior strap portions 3312 connect between a back strap portion 3334 and the respective headgear conduits 3350. In this example the superior strap portions 3312 connect to eyelets on tabs 3320 of the headgear conduits 3500. The inferior strap portions 3322 connect between the back strap portion 3334 and the plenum chamber 3200, in this example via headgear clips 3323.

The back strap portion 3334, superior strap portions 3312 and inferior headgear strap portions 3322 may be integrally formed. The headgear strap 3301 and its superior strap portions 3312, inferior headgear strap portions 3322 and back strap portion 3334 may be formed by a single flat knitting process.

In accordance with one aspect of the present technology, the various strap portions of the headgear strap 3301 may be joined using a stitchless joint 3370.

### 4.3.3.2.2 Conduit Headgear Tubes

In accordance with one aspect of the present technology, components of a conduit headgear 3301 may be joined using a stitchless joint 3370. As illustrated in FIG. 10B, the headgear conduits 3350 comprise a superior conduit portion 3362 and an inferior conduit portion 3363. In examples, a junction between the superior conduit portion 3362 and the inferior conduit portion 3363 may be provided using a stitchless joint 3370.

In further examples, the headgear conduits 3350 may be joined at a junction 3903 at a superior portion of the patient's head. In examples, the headgear conduits 3350 may be joined at a junction 3903 using stitchless joints 3370.

FIG. 10C shows an example of a conduit headgear 3301 having a similar structure to that described with reference to FIG. 3Y, and further having a flexible cover 3352 provided over at least a portion of each conduit 3350. The flexible cover 3352 may be provided for at least portions of each conduit 3350 which would otherwise contact the patient's face.

In examples, the flexible cover 3352 comprises a textile, for example a knitted textile. Circular knitting or 3D knitting may be used to form the flexible cover 3310.

In the example shown in FIG. 10C, a textile back strap 3334 may be formed separately from the cover 3352 and may be connected to the cover 3352, for example using stitchless joints 3370. Alternatively, strap connecting structures such as tabs 3320 (see, for example, FIG. 3Y and FIG. 10B) may be connected to the cover 3352.

In examples various components and structures of the conduit headgear 3301 may comprise textiles as described in PCT Patent Application No. PCT/AU2022/050773, the contents of which are incorporated herein by reference.

### 4.3.3.2.3 Imparting Properties to Headgear

In accordance with one aspect of the present technology, the at least one polymer layer may be used to impart different properties to the headgear.

In examples, the materials and/or construction of the polymer layer may be used to provide or control one or more of: water permeability, and/or air permeability. This may be particularly applicable to headgear straps.

In examples, the at least one polymer layer may increase stretchability of the headgear across the joint. In other examples, the at least one polymer layer may impart shape holding properties of the headgear. In further examples, the at least one polymer layer may impart increased rigidity to at least a portion of the headgear.

For example, the thickness of the at least one polymer layer may be selected or applied in order to achieve a desired characteristic. In other examples, the material(s) from which the at least one polymer layer is constructed may be selected in order to achieve a desired characteristic. In other examples, the shape and/or orientation of the at least one polymer layer in order to achieve a desired characteristic.

In one form of the technology, different regions of the headgear may comprise different properties in terms of stretch capabilities. One example of such headgear is disclosed in PCT Patent Publication No. WO2020261138A1, the contents of which are incorporated herein by reference. Another such example is disclosed in PCT Patent Publication No. WO2020170100A1, the contents of which are incorporated herein by reference.

In the example shown in FIG. 9A, the headgear straps of the positioning and stabilising structure 3300 may be stretchable. Advantageously, the superior strap portions 3312, inferior strap portions 3322 and ring strap portion 3340 are stretchable. The stretchable nature of the ring strap portion 3340 of the positioning and stabilising structure 3300 enables the ring strap portion 3340 to conform and fit snugly to the posterior, lateral and superior surfaces of the patient's head in use. Stretchiness in the superior strap portions 3312 and inferior strap portions 3322 enables these strap portions to extend in length slightly to provide some relief when the plenum chamber 3200 is pressurised. When the plenum chamber 3200 is under pressure in use, the volume of pressurised air inside the plenum chamber 3200 pushes the plenum chamber 3200 and frame 3500 in an anterior direction away from the patient's face. The force from this pressure must be countered by tension in the headgear straps in order to keep the plenum chamber 3200 and seal-forming structure 3100 in sealed contact with the patient's face. The ability of the superior strap portions 3312 and inferior strap portion 3322 to extend in length by at least a small amount can make wearing the patient interface 3000 more comfortable when this occurs. In examples this may be at least partially achieved using the stitchless joints 3370 of the present technology.

In examples, portions of the positioning and stabilising structure 3300 may have greater rigidity. For example, the back strap portion 3334 may be reinforced by the at least one polymer layer. This reinforcement may provide a high level of stability to the patient interface 3000 in use, since a purpose of the back strap portion 3334 is to provide an anchor for the other strap portions which connect to the plenum chamber 3200 while under tension to pull the plenum chamber 3200 towards the patient's face. The rigidised portion may be substantially non-stretchable or at least less stretchable than the other strap portions, although may still be bendable to conform to the curvature of the patient's head. The non-stretchable or low-stretch nature of the rigidised portion provides reinforcement to the back strap portion 3334, providing a firmer anchor and resulting in a more stable positioning and stabilising structure 3300.

In examples, the stitchless joint 3370 at the junction of the superior strap portions 3312 and the ring strap portion 3340 may be used to provide additional stiffness since, in use, the superior strap portions 3312 are under tension and there is a relatively large area of the strap material at the junction. Strengthening this junction may help provide a high level of stability to the patient interface 3000.

In the example shown in FIG. 9B, the crown strap portion 3330 may comprise relatively limited stretch capabilities or stretchability in order to provide stability on and around the crown of the patient's skull. For example, the crown strap portion 3330 has less stretchability (i.e. the amount of elongation per unit force) compared to some other portions of the headgear. The superior strap portion 3312, and anterior strap portions 3324 may also have limited stretch capabilities and preferably have less stretchability compared to some other portions of the headgear. The superior strap portion 3312 may have limited stretch capabilities in order to provide stability on and around the crown of the patient. The anterior strap portions 3324 may have limited stretch capabilities in order to hold the adjustment or provide stability at the interfacing portion. In other words, the crown strap portion 3330, the superior strap portion 3312, and the anterior strap portion 3324 may be relatively rigid compared to other straps of the headgear in order to maintain the shape of the headgear and assist in providing appropriate forces and comfort to the patient's head. Thus, the distance of elongation per unit force for the crown strap portion 3330, the superior strap portion 3312, and the anterior strap portion 3324 is less than other strap portions that comprise the headgear. The headgear may come in a variety of sizes (e.g., small, medium, large) in order to conform to patients with a variety of head sizes. In this way, the crown strap portion 3330, the superior strap portion 3312, and the anterior strap portion 3324 may not need to stretch, or otherwise deform, in order to meet size requirements for different patients. Instead, the crown strap portion 3330, the superior strap portion 3312, and the anterior strap portion 3324 may provide stability and/or rigidity regardless of the size of the individual patient's head.

The back strap portion 3334 may comprise a greater stretchability compared to the crown, superior and anterior strap portions. A greater stretchability in this area of the headgear allows the headgear to adjust to patients with different sized heads. The inferior strap portions 3322 may have relatively greater stretch capabilities (e.g., as compared to the superior and anterior strap portions 3312, the back strap portion 3334, etc.), preferably the most stretch compared to other portions of the headgear. Together, the inferior and back strap portions 3322, 3334 provide anterior/posterior flexion to the headgear, although the inferior strap portion 3322 may also provide some superior/inferior flexion (e.g., because the inferior strap portion 3322 extends in both the anterior/posterior and superior/inferior directions along the patient's head). In some embodiments, the headgear has sufficient elasticity to allow the headgear to be donned and doffed without undoing the strap attachment portion(s) 3305 or releasing the connection portions from the interfacing portion 3500. To don and doff the headgear the patient may pull the back of the headgear up and over the crown of the patient's head whilst the headgear is still connected to the interfacing portion 3500. The greater stretch capabilities of the back strap portion 3334 and the inferior strap portions 3322 may assist the patient in donning and doffing the headgear. In other words, the headgear (e.g., specifically the back strap portion 3334) may be pulled in the posterior direction, so that it does not directly apply tension to the patient's head. The gap created between the back strap portion 3334 and the patient's head permits the back strap portion 3334 to move in the superior direction along the patient's head, and eventually off of the patient's head. Stretching the back strap portion 3334 may also apply tension to the inferior strap portions 3322 (e.g., cause them to stretch). The stretching of the inferior strap portions 3322 may assist in manoeuvring the headgear around the patient's ears in order to minimize discomfort. Since the superior strap portion 3312 and the anterior strap portion 3324 may have little to no stretch capabilities (e.g., as compared to the inferior strap portion 3322), the opening for the ear will not completely deform and pinch the patient's ear as the patient is removing the headgear.

The posterior connecting portions 3336 may have a stretch capability between that of the inferior strap portion 3322 and the superior strap portion 3312. The posterior connecting portions 3336 may be disposed in the anterior/posterior and the superior/inferior directions, and may be able to provide extension in both directions.

The posterior connecting portions 3336 are preferably stiff enough to provide stability around the crown. Some or all of the posterior connecting portions 3336 may have a slightly greater stretch capability than the superior strap portion 3312 as these portions may need to stretch to assist when the patient is donning and doffing the headgear if the stretch of the inferior strap portion 3322 and/or the back strap portion 3334 is not enough to enable the headgear to clear the patient's head. In particular, the posterior connecting portions 3336 may provide extension mainly in the vertical direction, in order to provide additional extension to the mainly horizontal extension from the inferior and back strap portions 3334. In some embodiments, an inferior region of the posterior connecting portions 3336 comprises the greater stretch capability (e.g., than the rest of the posterior connecting portions 3336).

In some examples the region of the headgear with the greatest stretch capability is the area where the superior strap portions 3312, inferior strap portions 3322 and the back strap portion 3334 join. The combined stretch capabilities of these straps may provide this area with the ability to stretch to the greatest length. In other words, the confluence of the three stretchable straps allows the headgear to achieve the greatest amount of total combined length extension (e.g., in both the anterior/posterior and superior/inferior directions). One or more polymer layers may be used to influence this characteristic.

In some embodiments, the headgear may be formed of different segments with different stretch capabilities. The different segments may be joined together, for instance by using the stitchless joint 3370 of the present technology, to assist in achieving these capabilities.

In some forms, the superior back strap 3334a may be inextensible or substantially inextensible. Thus, the superior back strap 3334a may not be capable of stretching or extending the same amount as the inferior back strap 3334b.

In some forms, the inferior back strap 3334b may provide stretchability to the positioning and stabilizing structure 3300. Stretching across the inferior back strap 3334b may increase the distance between each inferior strap portion 3322, and/or a diameter of the opening. In either case, stretching across the inferior back strap 3334b may assist in allowing patients with different sized heads comfortably don and doff the positioning and stabilizing structure 3300. The inferior back strap 3334b may be returned to substantially its original length when a force is no longer applied so that the positioning and stabilizing structure 3300 remains substantially snug on the patient's head (e.g., automatic adjustments), and can be worn repeatedly without damaging (e.g., permanently deforming) the elastic material.

Accordingly, a central section (e.g., positioned to contact the patient's head proximate the occiput) of the back strap portion 3334 may stretch less than sides of the back strap portion 3334. In certain forms, the connections between the inferior back strap 3334b and the inferior strap portion 3322 may stretch, while the central region of the inferior back strap 3334b is held relatively stiff by the connection to the superior back strap 3334a. The total length of extension may be less (e.g., as compared to an unconnected inferior back strap 3334b), but the inferior back strap 3334b may still be able to stretch to accommodate various sizes of heads.

In an example, a portion of the back strap portion 3334 may be rigidised, such as with an increased thickness and/or more rigid polymer layer. The rigidised portion may reinforce the back strap portion 3334. This reinforcement may provide a high level of stability to the patient interface 3000 in use, since a purpose of the back strap portion 3334 is to provide an anchor for the other strap portions which connect to the plenum chamber 3200 while under tension to pull the plenum chamber 3200 towards the patient's face. The rigidised portion may be substantially non-stretchable or at least less stretchable than the other strap portions, although may still be bendable to conform to the curvature of the patient's head. The non-stretchable or low-stretch nature of the rigidised portion provides reinforcement to the back strap portion 3334, providing a firmer anchor and resulting in a more stable positioning and stabilising structure 3300.

In the example shown in FIG. 10A, at least a portion of the back strap portion 3334 of the headgear strap 3301 is rigidised in order to reinforce the back strap portion 3334. This reinforcement may provide a high level of stability to the patient interface 3000 in use, since a purpose of the back strap portion 3334 is to provide an anchor for the other strap portions which connect to the plenum chamber 3200 while under tension to pull the plenum chamber 3200 towards the patient's face. The rigidised portion may be substantially non-stretchable or at least less stretchable than the other strap portions, although may still be bendable to conform to the curvature of the patient's head. The superior strap portions 3312 and inferior strap portions 3322 may be stretchable, and/or the stitchless joints 3370 may provide a degree of stretch relative to the back strap portion 3334.

### 4.3.3.2.4 Surface Finishing

According to one aspect of the present technology, the at least one polymer layer may be provided with one or more surface finishes in accordance with desired aesthetic and/or functional characteristics or properties.

In examples, the surface finish may comprise one or more of: colouring, luminescence, pattern, shapes, texture, markings.

In examples, at least one characteristic of the surface finish may be substantially the same as a surface finish of material to which the at least one polymer layer is applied. For example, the surface finish may be selected to substantially match that of an adjacent surface in order to provide a substantially continuous feel to a user. In alternative examples the surface finish may be different to that of the material to which the at least one polymer layer is applied.

In examples, the surface finish may be provided to the at least one polymer layer prior to production of the stitchless joint. In alternative examples the surface finish may be provided to the at least one polymer layer after production of the stitchless joint.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 4.3.5 Decoupling Structure(s)

In one form the patient interface 3000 includes at least one decoupling structure, for example, a swivel or a ball and socket.

### 4.3.6 Connection Port

Connection port 3600 allows for connection to the air circuit 4170.

### 4.3.7 Forehead Support

In one form, the patient interface 3000 includes a forehead support 3700.

### 4.3.8 Anti-Asphyxia Valve

In one form, the patient interface 3000 includes an anti-asphyxia valve.

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.4.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6s, peak inspiratory flow rate Qpeak 0.4 L/s, exhalation time Te 2.4s, peak expiratory flow rate Qpeak-0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

### 4.8 Respiratory Therapy Modes

Various respiratory therapy modes may be implemented by the disclosed respiratory therapy system.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.9.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.9.2 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Functional dead space: (description to be inserted here)

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A positioning and stabilising structure to provide a force to hold a seal-forming structure of a patient interface in a therapeutically effective position on a patient's head, the seal-forming structure constructed and arranged to form a seal with a region of the patient's face surrounding an entrance to the patient's airways for sealed delivery of a flow of air at a therapeutic pressure of at least 6 cmH2O above ambient air pressure to at least the patient's nares throughout the patient's respiratory cycle in use, the positioning and stabilising structure comprising:
a headgear portion, the headgear portion comprising a first headgear portion and a second headgear portion, wherein the first headgear portion and the second headgear portion are joined by a stitchless joint comprising at least one polymer layer spanning between the first headgear portion and the second headgear portion and applied to the first headgear portion and the second headgear portion.

2. The positioning and stabilising structure of claim 1, wherein adjacent edges of the first headgear portion and the second headgear portion joined by the stitchless joint are non-overlapping.

3. The positioning and stabilising structure of claim 2, wherein the adjacent edges of the first headgear portion and the second headgear portion joined by the stitchless joint are abutting when the positioning and stabilising structure is unloaded.

4. The positioning and stabilising structure of claim 2, wherein the adjacent edges of the first headgear portion and the second headgear portion are directly joined together in addition to the at least one polymer layer.

5. The positioning and stabilising structure of claim 4, wherein the adjacent edges of the first headgear portion and the second headgear portion are welded together.

6. The positioning and stabilising structure of claim 2, wherein the adjacent edges of the first headgear portion and the second headgear portion are not directly joined.

7. The positioning and stabilising structure of claim 1, wherein the first headgear portion and the second headgear portion are strap portions.

8. The positioning and stabilising structure of claim 1, wherein the first headgear portion and the second headgear portion are headgear conduit portions.

9. The positioning and stabilising structure of claim 1, wherein one of the first headgear portion and the second headgear portion is a strap portion, and the other one of the first headgear portion and the second headgear portion is a headgear conduit portion.

10. The positioning and stabilising structure of claim 1, wherein one of the first headgear portion and the second headgear portion is a strap connection tab portion, and the other one of the first headgear portion and the second headgear portion is a headgear conduit portion.

11. The positioning and stabilising structure of claim 1, wherein the at least one polymer layer surrounds the first headgear portion and the second headgear portion at the stitchless joint.

12. The positioning and stabilising structure of claim 1, wherein the at least one polymer layer comprises a first polymer layer applied to a first side of the first headgear portion and the second headgear portion, and a second polymer layer applied to a second side of the first headgear portion and the second headgear portion.

13. (canceled)

14. (canceled)

15. (canceled)

16. (canceled)

17. (canceled)

18. The positioning and stabilising structure of claim 1, wherein the at least one polymer layer overlaps each of the first headgear portion and the second headgear portion by a minimum distance to resist a tension force of at least 20 N across the joint.

19. (canceled)

20. The positioning and stabilising structure of claim 1, wherein the at least one polymer layer overlaps each of the first headgear portion and the second headgear portion by a minimum distance to resist a tension force of at least 40 N across the joint.

21. (canceled)

22. (canceled)

23. (canceled)

24. The positioning and stabilising structure of claim 1, wherein the at least one polymer layer imparts shape holding properties of the headgear.

25. The positioning and stabilising structure of claim 1, wherein the at least one polymer layer imparts increased rigidity to at least a portion of the headgear.

26. (canceled)

27. The positioning and stabilising structure of claim 1, wherein the positioning and stabilising structure comprises:
at least one gas delivery tube to receive the flow of air from a connection port on top of the patient's head and to deliver the flow of air to the entrance of the patient's airways via the seal-forming structure, the gas delivery tube being constructed and arranged to contact, in use, at least a region of the patient's head superior to an otobasion superior of the patient's head,
wherein the at least one gas delivery tube comprises a pair of headgear conduits to receive the flow of air from a connection port on top of the patient's head and to deliver the flow of air to the entrance of the patient's airways via the seal-forming structure, each headgear conduit constructed and arranged to contact, in use, at least a region of the patient's head superior to an otobasion superior of the patient's head on a respective side of the patient's head; and
a headgear strap.

28. The positioning and stabilising structure of claim 27, wherein a stitchless joint comprising at least one polymer layer is provided between a portion of the headgear conduit and a portion of the headgear strap.

29. (canceled)

30. The positioning and stabilising structure of claim 27, comprising a flexible cover provided to at least a portion of each headgear conduit.

31. (canceled)

32. (canceled)

33. (canceled)

34. A patent interface, comprising:
a seal-forming structure constructed and arranged to form a seal with a region of a patient's face surrounding an entrance to the patient's airways for sealed delivery of a flow of air at a therapeutic pressure to at least the patient's nares throughout the patient's respiratory cycle in use; and
a positioning and stabilising structure to provide a force to hold the seal-forming structure in a therapeutically effective position on the patient's head, the positioning and stabilising structure comprising:
a headgear portion, the headgear portion comprising a first headgear portion and a second headgear portion, wherein the first headgear portion and the second headgear portion are joined by a stitchless joint comprising at least one polymer layer spanning between the first headgear portion and the second headgear portion and applied to the first headgear portion and the second headgear portion.
